(19) United States
(12) Patent Application Publication
(10) Pub. No.: US 2026/0096744 A1
(43) Pub. Date: Apr. 9, 2026

(54) SYSTEM AND METHOD FOR MOISTURE VAPOR TRANSFER

(71) Applicant: Pentair, Inc., Minneapolis, MN (US)

(72) Inventors: Sarah Farley, White Bear Lake, MN (US); Robert Orvin Crowder, Lino Lakes, MN (US); Mohan R. Khadilkar, Plymouth, MN (US)

(21) Appl. No.: 19/115,152

(22) PCT Filed: Oct. 2, 2023
(86) PCT No.: PCT/US2023/075753

§ 371 Date: Mar. 25, 2025

Related U.S. Application Data
(60) Provisional application No. 63/377,985, filed on Sep. 30, 2022.

(51) Int. Cl. A61B 5/097; A61B 5/083; B01D 53/26; B01D 53/28; B01J 20/26; C08G 18/40; G01N 33/497

(52) U.S. Cl. CPC A61B 5/097; A61B 5/083; B01D 53/261; B01D 53/28; B01J 20/262; C08G 18/4018; G01N 33/497; B01D 2253/202; B01D 2257/80; B01D 2259/4533

(57) ABSTRACT

A system and a method for removing moisture from a fluid are provided. The system includes a source of a fluid, the fluid imparted with a first concentration of water, and a first conduit in communication with the source of the fluid. The apparatus further includes a polymeric material provided in the form of an elongated cylinder defining a sidewall. A first end of the elongated cylinder is coupled to the first conduit. The polymeric material is adapted to remove water from the fluid to create a dehydrated fluid sample imparted with a second concentration of water. The apparatus also comprises a second conduit which is coupled to the second end of the elongated cylinder and is in fluid communication with an outlet.

### CROSS-REFERENCE TO RELATED APPLICATIONS

This application claims priority to U.S. Provisional Patent Application Ser. No. 63/377,985, filed on Sep. 30, 2022, entitled “MOISTURE VAPOR TRANSFER POLYMERIC TUBE,” currently pending, the entire disclosure of which is incorporated herein by reference.

### FIELD OF DISCLOSURE

The present disclosure relates to systems and methods for removing moisture from a fluid. More particularly, the present disclosure relates to systems and methods for removing water from a fluid using a polymeric material.

### BACKGROUND

Patients suffering from a wide variety of medical conditions exhibit changes in the percentage of carbon dioxide (CO₂) and other gases emitted via metabolic processes in their breath. Breath analysis techniques, for example, capnography, can be used to measure carbon dioxide content in exhaled breath. Monitoring the percentage of carbon dioxide and other gases in the patient's breath can deliver healthcare providers valuable information regarding patient health and provide a warning in the event of physiological changes. One common problem with breath analysis is that moisture in the breath sample interferes with the ability of analyzers to detect gases accurately, which necessitates a pre-conditioning of the sample breath via drying before the analysis can occur.

In various breath analysis systems, breath gas can be sampled by a mainstream or a sidestream analyzer. In mainstream analyzers, the breath sample chamber is typically positioned within the flow path of the patient's gas stream, usually near the end of the respiratory interface.

In sidestream analyzers, the breath sample for analysis is drawn away from the flow path of the patient's gas stream by a tube. The tube, which may be connected to an adapter, delivers the breath sample to a breath sample chamber. To help prevent issues with the analyzer, the sample chamber should be free from moisture, including condensed liquids. Condensed liquids are generally water that condenses out from the humidity (e.g., the water vapor in breath) in the sampling tubes. The internal humidity levels in the sidestream tubes are high because of the proximity to the breath collection area since the exhaled and inhaled breath is humid and relatively warm. Condensed liquids are a problem commonly hindering breath analysis, particularly sidestream capnography.

The gas concentrations of the patient's breathing gases are monitored by transferring a portion of the breathing gases through a sampling line to a suitable gas monitor. The patient's breathing gases are usually saturated with moisture at body temperature. As the gas sample passes through the sampling line and cools, water naturally condenses out of the breathing gas. Collected condensate, along with secretions, bacteria, and other contaminants may result in inaccurate gas concentration readings or even adversely affect a delicate gas monitor.

Additionally, the respiratory interface and sampling line(s) should be manufactured in a way that allows for undistorted gas sample flow. Distortion of the gas sample flow (e.g., generation of non-laminar sample gas flow), regardless of cause, can alter or degrade the rise time of the measured waveform making accurate analysis, especially at higher breath rates, difficult or impossible.

A variety of different types of membranes have been described for use in removing moisture from breath gases. The polymeric materials used to prepare these membranes include polymers with polyether and polyamide segments (i.e., polyethylene oxide) and hydrophilic organic polymers such as polyvinyl alcohol, polyimides, polyamides, and polyelectrolytes. In addition, inorganic materials such as molecular sieves and minerals (e.g., zeolites) having a microporous structure have been used. Other systems include a water trap, or another moisture separation means.

Moreover, moisture permeable tubes have been used for breath analysis applications (such as capnography). These tubes exhibit high permeability to moisture but do not readily pass other respiratory gases, such as oxygen and carbon dioxide. When used in breath analysis, typical moisture permeable tubes cannot be used for multiple patients and cannot be re-used for the same patient. The disposable nature of the typical moisture permeable tubes increases the cost factor. The cost becomes even more significant in applications that require relatively long tubes. Additionally, the structural supports complicate the manufacture of the product and reduce the water permeation.

There are several additional disadvantages to using these moisture permeable tubes to remove moisture from fluids, such as breath gases. For example, many polymer materials including polyether and polyamide segments are brittle, vulnerable to bond breakage of ester links and unstable when exposed to water, which is exacerbated at high temperatures.

Another disadvantage of the current polymeric materials is the chemically aggressive nature of the raw materials used for their preparation and the difficulty in the processing of these materials. Further, integrating the polymers into tubing systems is complicated and requires special means.

Thus, there is a need for improved materials and apparatuses exhibiting moisture permeable properties, which are effective, easy to handle and manufacture, and cost-efficient.

Thus, there is a need to provide an improved apparatus that is easily removed, mounted from one system to another system and wherein the apparatus can remove moisture from a fluid to enable accurate analysis of the fluid. Also, there is a need for improvement of prior art solutions for respiratory gas analysis in respect of, e.g., low distortion, long-lasting moisture and/or water removal, or low cost.

### SUMMARY

In one aspect, an apparatus is provided in the form of a source of a fluid imparted with a first concentration of water, a first conduit, a second conduit, and a polymeric material. The first conduit is in fluid communication with the source of the fluid. The polymeric material is provided having a first end coupled to the first conduit and a second end opposite the first end. A channel extends through the sidewall from the first end and to the second end, and the polymeric material is adapted to remove water from the fluid to create a dehydrated fluid imparted with a second concentration of water. The second conduit is coupled to the second end of the polymeric material, and the second conduit is also in fluid communication with an outlet.

In some embodiments, the apparatus includes an analyzer in fluid communication with the second conduit, and the analyzer is configured to measure at least one parameter of the dehydrated fluid.

In other embodiments, the apparatus includes a sweep gas that flows over the polymeric material, wherein the sweep gas transfers water away from the polymeric material.

In yet other embodiments, the source of the fluid in the apparatus is selected from the group consisting of a breath gas, a hydraulic fluid, a lubrication oil, a transformer oil, and a liquid fuel.

In some embodiments, the apparatus includes a polymeric material wherein the polymeric material comprises a block copolymer including a polyurethane block copolymer.

In other embodiments, the apparatus includes a polymeric material defined by a first polymer and a second polymer, and in which the first polymer is at least one of (a) a block copolymer including a polyether, or (b) a polyester polyether block copolymer (COPE).

In another aspect, an apparatus for removing moisture from a fluid is provided. The apparatus includes a polymeric material provided in the form of a body, the body shaped in the form of a tube, and a source of a water-containing fluid. The source is in fluid communication with the polymeric material, and the water-containing fluid is imparted with a first moisture value. The polymeric material is adapted to remove moisture from the water-containing fluid to create a second fluid imparted with a second moisture value. In some instances, the second moisture value is less than the first moisture value.

In other embodiments, the apparatus includes a polymeric material that comprises a polymer composition including two or more prepolymers. The prepolymers include at least one of diisocyanate blocks connected by urethane linkages to diols of variable chain length or a first alcohol or a carboxylic acid homopolymer block covalently bonded to a second alcohol or carboxylic acid homopolymer.

In yet other embodiments, the apparatus includes a gas analyzer, and the gas analyzer measures at least one parameter of the second fluid.

In some embodiments, the apparatus includes a polymeric material in which the polymeric material comprises a block copolymer including a polyurethane block copolymer.

In other embodiments, the water-containing fluid in the apparatus is a breath gas, and the breath gas is obtained from a patient.

In yet other embodiments, the apparatus includes a polymeric material provided in the form of a blend of polymers, the composition including at least one of a polyurethane block copolymer or a polyester polyether block copolymer (COPE).

In some embodiments, the apparatus includes a polymeric material comprising a polymer composition including two or more prepolymers, and the prepolymers include at least one of diisocyanate blocks connected by urethane linkages to diols of variable chain length or an alcohol or a carboxylic acid homopolymer block covalently bonded to another alcohol or carboxylic acid homopolymer.

In another aspect, a method of removing moisture from a fluid is provided. The method comprises the steps of collecting a water-containing fluid sample and directing the water-containing fluid through a polymeric material to remove water from the water-containing fluid. Directing the water-containing fluid through the polymeric material creates a dehydrated fluid sample at least partially free of water contamination. The method also comprises the step of directing the dehydrated fluid sample out of the polymeric material and to an analyzer. The method further comprises the step of evaluating the dehydrated fluid sample using the analyzer.

In some embodiments, the method of using an apparatus creates a dehydrated fluid sample that is substantially free of water contamination.

In other embodiments, the polymer composition of the polymeric material in the method of using an apparatus includes a first polymer, where the first polymer is selected from a polyurethane block copolymer, a polyether block copolymer, a polyester polyether block copolymer (COPE), a diisocyanate block connected by urethane linkages to diols of variable chain length, a first alcohol or a carboxylic acid homopolymer block covalently bonded to a second alcohol or carboxylic acid homopolymer, or combinations thereof.

In yet other embodiments, the water-containing fluid in the method of using an apparatus includes a water-containing breath gas, and the method further includes the steps of sampling water-containing breath gases of a patient by attaching a respiratory interface to the patient and placing an outlet of the respiratory interface into fluid communication with the polymeric material.

In some embodiments, the analyzer is configured to measure a concentration of at least one of oxygen, carbon dioxide, carbon monoxide, or nitrogen of the dehydrated fluid sample.

In other embodiments, the polymer composition of the polymeric material includes at least a block copolymer comprising a polyester polyether block copolymer (COPE).

### DESCRIPTION OF THE DRAWINGS

FIG. 1 is a schematic representation of an apparatus including a polymeric material adapted to remove moisture from a fluid;

FIG. 2 is a side isometric view of a polymeric material adapted to remove moisture from a fluid;

FIG. 3 is a partial side view of a subject connected to a respiratory interface system, the respiratory interface system in fluid communication with the polymeric material of FIG. 2 and adapted to remove moisture from a breath gas of the subject;

FIG. 4 is a schematic representation of the apparatus of FIG. 3;

FIG. 5 is an isometric view of an oil-conditioning apparatus including a polymeric material, the polymeric material adapted to remove moisture from an oil provided to the oil-conditioning apparatus;

FIG. 6 is a schematic representation of a method of removing water from a fluid sample; and

FIG. 7 is a schematic representation of a method of removing water from breath gases.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the disclosure. Various modifications to the illustrated embodiments will be readily apparent to those skilled in the art, and the generic principles herein can be applied to other embodiments and applications without departing from embodiments of the disclosure. Thus, embodiments of the disclosure are not intended to be limited to embodiments shown but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the disclosure. Skilled artisans will recognize the examples provided herein have many useful alternatives and fall within the scope of embodiments of the disclosure.

According to the teachings herein, an apparatus and a method of using the apparatus is provided. The apparatus comprises a polymeric material provided in the form of an elongated, substantially hollow cylinder which is designed to remove moisture from a water-containing fluid (i.e., a water-containing liquid or gas). For example, the apparatus may remove moisture from fluids provided as breath gases, hydraulic fluids, lubrication oils, transformer oils, and liquid fuels. The apparatus may be coupled to an analyzer, such as a gas analyzer, which may provide information regarding the composition of the dehydrated fluid.

As described herein, the apparatus may allow for moisture in a gas sample to permeate through a polymeric material to provide a dehydrated gas sample, while other gaseous components (e.g., nitrogen, oxygen, carbon dioxide) of the gas sample pass through the polymeric material substantially without permeation. The removal of the water from the gas sample may allow for a more accurate downstream analysis of the dehydrated sample.

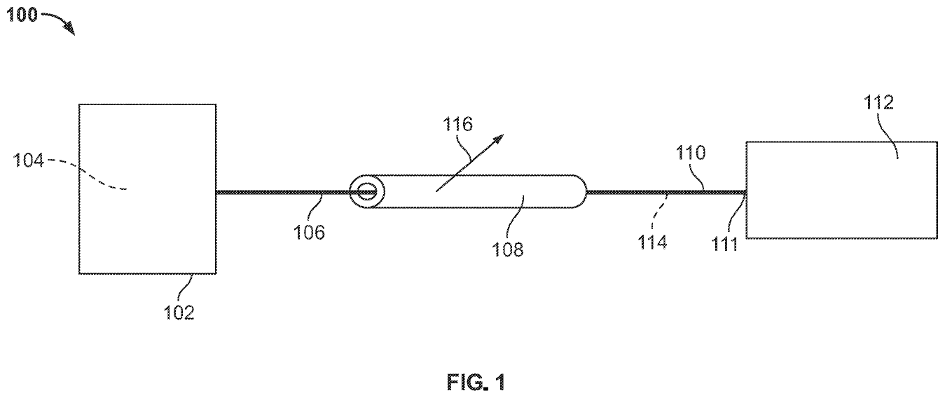

Referring now to FIG. 1, a fluid dehydrating apparatus 100 is provided in the form of a source 102, a fluid 104 imparted with a first concentration of water, a first conduit 106, a polymeric material 108, a second conduit 110, and an analyzer 112, each of which are in fluid communication with respect to each other. The apparatus 100 may be configured to remove moisture from the fluid 104 using the polymeric material 108.

The apparatus 100 may be used for sampling a stream of gas and transporting it to the analyzer 112. In some embodiments, the apparatus 100 may be adapted for a wide range of applications, including gas sampling from intubated patients, and nasal or oral gas sampling. In some embodiments, the apparatus can be adapted for patients having a high breath rate, such as infants, children, or other patients with weak respiration.

Generally, the source 102 provides a water-containing fluid 104 imparted with a first concentration of water (e.g., the fluid 104) to the polymeric material 108. For example, the source 102 may be a respiratory apparatus, a patient, a source of hydraulic fluid, a source of lubrication oil, a source of transformer oil, and/or a source of liquid fuel.

The fluid 104 may be provided as breath gas, a hydraulic fluid, a lubrication oil, a transformer oil, or a liquid fuel.

The first conduit 106 may be provided in the form of any structure having a channel for conveying fluid. In some aspects, the first conduit 106 may be a tube for conveying fluid. The first conduit 106 may be coupled to the source 102 using various fittings including using screws or compression, threaded couplings screwing into couplings with threaded ends, conduit straps, and/or other similar devices used in the art.

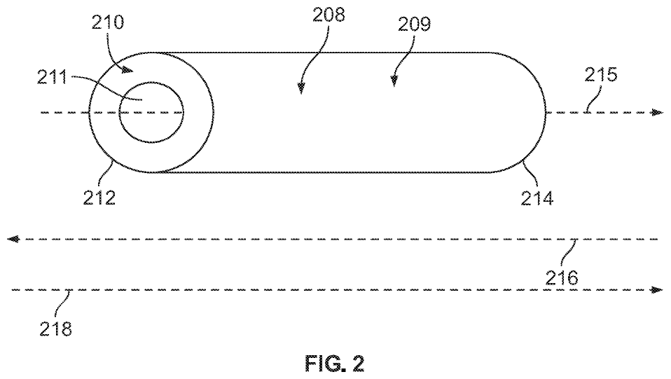

The polymeric material 108 of the apparatus 100 may be provided in many shapes and forms configured to transport the fluid 104 and remove moisture from the fluid 104. For example, the polymeric material may be provided as a body 209 shaped as an elongated cylinder (e.g., see FIG. 2).

The polymeric material 108 is adapted to remove water 116 from the fluid 104 to create a dehydrated fluid 114 imparted with a second concentration of water, which may be transferred through the second conduit 110 and to an outlet 111. The outlet 111 may be in fluid communication with additional components of the apparatus 100 (e.g., the analyzer 112) and/or an outside environment. Alternatively, or in addition, the dehydrated fluid 114 may be transferred through the second conduit 110 to the analyzer 112 in fluid communication with the second conduit 110 (e.g., via the outlet 111).

The analyzer 112 is configured to measure at least one parameter of the dehydrated fluid 114 (e.g., the concentration of non-water components of the dehydrated fluid 114). The analyzer 112 may be any analyzer capable of measuring analytes in the dehydrated sample 114 including but not limited to oxygen, carbon dioxide, carbon monoxide, and/or nitrogen. The analyzer 112 may be provided in the form of an infrared spectrometer, a UV-Vis spectrometer, a Raman spectrometer, gas chromatography system, or a mass spectrometer.

The various embodiments of the apparatus 100 have further advantages as the apparatus 100 may remove moisture from the gases or liquids without any significant loss of the desired analytes from the fluid 104. Additionally, in some embodiments, the moisture vapor transfer from the gases or liquids provided to the polymeric material 108 improves analytical outcomes and protects equipment from degradation.

The polymeric material 108 may be provided in the form of a moisture permeable polymer composition. In some embodiments, the polymer composition may comprise a base component of Formula I.

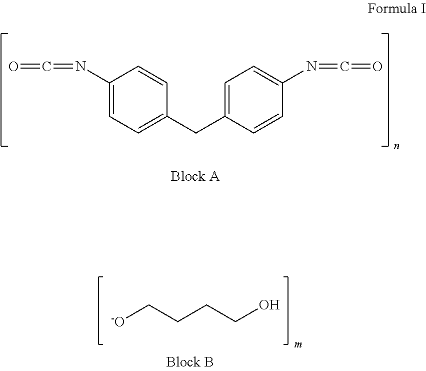

In other embodiments, the polymer composition may include two or more prepolymers, wherein the prepolymers include at least one of: (a) diisocyanate blocks connected by urethane linkages to diols of variable chain length or (b) an alcohol or a carboxylic acid homopolymer block covalently bonded to another alcohol or carboxylic acid homopolymer.

In some embodiments, the polymer composition may comprise a block copolymer including a polyurethane block copolymer. In some embodiments, the polymer composition may be in the form of a polymeric material comprising a polyester polyether block copolymer (COPE). In one embodiment, the polymer composition comprises a base component of Formula II.

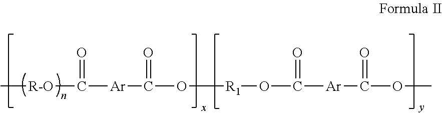

Additionally, some embodiments of the polymeric material 108 include various blends of thermoplastic urethanes (TPU) and thermoplastic polymers such as copolyester-ester or ether polymers (COPE). Some embodiments of polymeric material 108 comprise a first polymer and a second polymer, wherein the first polymer includes at least one of a polyurethane block copolymer or a polyester polyether block copolymer (COPE). In various embodiments, the TPU included in the polymeric material 108 can be provided as an aliphatic polyether type polymer. In other embodiments, the TPU included in the polymeric material 108 can be an aliphatic blend polymer. In various embodiments of the polymeric material 108, the COPE polymer can be a polyester polyether type polymer.

The polymeric material 108 may be provided as a polymer composition including a first polymer. The first polymer may be selected from the group consisting of a polyurethane block copolymer, a polyether block copolymer, a polyester polyether block copolymer (COPE), a diisocyanate block connected by urethane linkages to diols of variable chain length, a first alcohol or a carboxylic acid homopolymer block covalently bonded to a second alcohol or carboxylic acid homopolymer, and combinations thereof. As an additional example, the polymeric material 108 may also comprise a blend of polymers including various blends of thermoplastic urethanes (TPU) and thermoplastic polymers such as copolyester-ester or ether polymers (COPE). Some embodiments of the polymeric material 108 comprise a first polymer and a second polymer, wherein the first polymer includes at least one of a polyurethane block copolymer or a polyester polyether block copolymer (COPE). In various embodiments, the TPU included in the polymeric material 108 can be an aliphatic polyether type polymer. In other embodiments, the TPU included in the polymeric material 108 can be an aliphatic blend polymer. In various embodiments of the polymeric material 108, the COPE polymer can be a polyester polyether type polymer.

In some instances, the polymeric material 108 may be provided as one or more polymers. In such instances, each polymer of the one or more polymers may be provided as any polymer described herein.

The polymeric material 108 may be provided as a blend of polymers. For example, the blend of polymers may include at least two polymers (e.g., a first polymer and a second polymer). The first and second polymers may be any polymer that is described herein. In some instances, the first polymer may comprise the polymer blend in an amount ranging from about 0% wt/wt to about 100% wt/wt. In other instances, the second polymer may comprise the polymer blend in an amount ranging from about 0% wt/wt to about 100% wt/wt. In some instances, the first polymer may comprise the polymer blend in an amount ranging from 0% wt/wt to 100% wt/wt. In other instances, the second polymer may comprise the polymer blend in an amount ranging from 0% wt/wt to 100% wt/wt.

Yet another embodiment includes a polymer composition that comprises at least about 50% wt/wt of the first polymer and no more than about 50% wt/wt of the second polymer. In another embodiment, the polymer composition comprises at least about 60% wt/wt of the first polymer and no more than about 40% wt/wt of the second polymer. In another embodiment, the polymer composition comprises at least about 70% wt/wt of the first polymer and no more than about 30% wt/wt of the second polymer. In another embodiment, the polymer composition comprises at least about 80% wt/wt of the first polymer and no more than about 20% wt/wt of the second polymer. In another embodiment, the polymer composition comprises at least about 90% wt/wt of the first polymer and no more than about 10% wt/wt of the second polymer. In another embodiment, the polymer composition comprises at least about 40% wt/wt of the first polymer and no more than about 60% wt/wt of the second polymer. In another embodiment, the polymer composition comprises at least about 30% wt/wt of the first polymer and no more than about 70% wt/wt of the second polymer. In another embodiment, the polymer composition comprises at least about 20% wt/wt of the first polymer and no more than about 80% wt/wt of the second polymer. In another embodiment, the polymer composition comprises at least about 10% wt/wt of the first polymer and no more than about 90% wt/wt of the second polymer.

Yet another embodiment includes a polymer composition which comprises 50% wt/wt of the first polymer and 50% wt/wt of the second polymer. In another embodiment, the polymer composition comprises at least 60% wt/wt of the first polymer and no more than 40% wt/wt of the second polymer. In another embodiment, the polymer composition comprises at least 70% wt/wt of the first polymer and no more than 30% wt/wt of the second polymer. In another embodiment, the polymer composition comprises at least 80% wt/wt of the first polymer and no more than 20% wt/wt of the second polymer. In another embodiment, the polymer composition comprises at least 90% wt/wt of the first polymer and no more than 10% wt/wt of the second polymer. In another embodiment, the polymer composition comprises at least 40% wt/wt of the first polymer and no more than 60% wt/wt of the second polymer. In another embodiment, the polymer composition comprises at least 30% wt/wt of the first polymer and no more than 70% wt/wt of the second polymer. In another embodiment, the polymer composition comprises at least 20% wt/wt of the first polymer and no more than 80% wt/wt of the second polymer. In another embodiment, the polymer composition comprises at least 10% wt/wt of the first polymer and no more than 90% wt/wt of the second polymer.

Yet another embodiment includes a polymer composition which comprises about 50% wt/wt of a TPU and about 50% wt/wt of a COPE polymer. In another embodiment, the polymer composition comprises at least about 60% wt/wt of a TPU and no more than about 40% wt/wt of a COPE polymer. In another embodiment, the polymer composition comprises at least about 70% wt/wt of a TPU and no more than about 30% wt/wt of a COPE polymer. In another embodiment, the polymer composition comprises at least about 80% wt/wt of a TPU and no more than about 20% wt/wt of a COPE polymer. In another embodiment, the polymer composition comprises at least about 90% wt/wt of a TPU and no more than about 10% wt/wt of a COPE polymer. In another embodiment, the polymer composition comprises at least about 40% wt/wt of a TPU and no more than about 60% wt/wt of a COPE polymer. In another embodiment, the polymer composition comprises at least about 30% wt/wt of a TPU and no more than about 70% wt/wt of a COPE polymer. In another embodiment, the polymer composition comprises at least about 20% wt/wt of a TPU and no more than about 80% wt/wt of a COPE polymer. In another embodiment, the polymer composition comprises at least about 10% wt/wt of a TPU and no more than about 90% wt/wt of a COPE polymer.

Yet another embodiment includes a polymer composition that comprises 50% wt/wt of a TPU and 50% wt/wt of a COPE polymer. In another embodiment, the polymer composition comprises at least 60% wt/wt of a TPU and no more than 40% wt/wt of a COPE polymer. In another embodiment, the polymer composition comprises at least 70% wt/wt of a TPU and no more than 30% wt/wt of a COPE polymer. In another embodiment, the polymer composition comprises at least 80% wt/wt of a TPU and no more than 20% wt/wt of a COPE polymer. In another embodiment, the polymer composition comprises at least 90% wt/wt of a TPU and no more than 10% wt/wt of a COPE polymer. In another embodiment, the polymer composition comprises at least 40% wt/wt of a TPU and no more than 60% wt/wt of a COPE polymer. In another embodiment, the polymer composition comprises at least 30% wt/wt of a TPU and no more than 70% wt/wt of a COPE polymer. In another embodiment, the polymer composition comprises at least 20% wt/wt of a TPU and no more than 80% wt/wt of a COPE polymer. In another embodiment, the polymer composition comprises at least 10% wt/wt of a TPU and no more than 90% wt/wt of a COPE polymer.

Some embodiments include a polymer composition that comprises about 50% wt/wt of a first TPU and about 50% wt/wt of a second TPU. In another embodiment, the polymer composition comprises at least about 70% wt/wt of a first TPU and no more than about 30% wt/wt of a second TPU polymer. In another embodiment, the polymer composition comprises at least about 80% wt/wt TPU and no more than about 20% wt/wt of a second TPU. In another embodiment, the polymer composition comprises at least about 90% wt/wt of a first TPU and no more than about 10% wt/wt of a second TPU. In another embodiment, the polymer composition comprises at least about 40% wt/wt of a first TPU and no more than about 60% wt/wt of a second TPU. In another embodiment, the polymer composition comprises at least about 30% wt/wt of a first TPU and no more than about 70% wt/wt of a second TPU. In another embodiment, the polymer composition comprises at least about 20% wt/wt of a first TPU and no more than about 80% wt/wt of a second TPU. In another embodiment, the polymer composition comprises at least about 10% wt/wt of a first TPU and no more than about 90% wt/wt of a second TPU.

Some embodiments include a polymer composition that comprises 50% wt/wt of a first TPU and 50% wt/wt of a second TPU. In another embodiment, the polymer composition comprises at least 70% wt/wt of a first TPU and no more than 30% wt/wt of a second TPU polymer. In another embodiment, the polymer composition comprises at least 80% wt/wt TPU and no more than 20% wt/wt of a second TPU. In another embodiment, the polymer composition comprises at least 90% wt/wt of a first TPU and no more than 10% wt/wt of a second TPU. In another embodiment, the polymer composition comprises at least 40% wt/wt of a first TPU and no more than 60% wt/wt of a second TPU. In another embodiment, the polymer composition comprises at least 30% wt/wt of a first TPU and no more than 70% wt/wt of a second TPU. In another embodiment, the polymer composition comprises at least 20% wt/wt of a first TPU and no more than 80% wt/wt of a second TPU. In another embodiment, the polymer composition comprises at least 10% wt/wt of a first TPU and no more than 90% wt/wt of a second TPU.

Some embodiments include a polymer composition that comprises about 50% wt/wt of a first COPE polymer and about 50% wt/wt of a second COPE polymer. In another embodiment, the polymer composition comprises at least about 70% wt/wt of a first COPE polymer and no more than about 30% wt/wt of a second COPE polymer. In another embodiment, the polymer composition comprises at least about 80% wt/wt COPE polymer and no more than about 20% wt/wt of a second COPE polymer. In another embodiment, the polymer composition comprises at least about 90% wt/wt of a first COPE polymer and no more than about 10% wt/wt of a second COPE polymer. In another embodiment, the polymer composition comprises at least about 40% wt/wt of a first COPE polymer and no more than about 60% wt/wt of a second COPE polymer. In another embodiment, the polymer composition comprises at least about 30% wt/wt of a first COPE polymer and no more than about 70% wt/wt of a second COPE polymer. In another embodiment, the polymer composition comprises at least about 20% wt/wt of a first COPE polymer and no more than about 80% wt/wt of a second COPE polymer. In another embodiment, the polymer composition comprises at least about 10% wt/wt of a first COPE polymer and no more than about 90% wt/wt of a second COPE polymer.

Some embodiments include a polymer composition that comprises 50% wt/wt of a first COPE polymer and 50% wt/wt of a second COPE polymer. In another embodiment, the polymer composition comprises at least 70% wt/wt of a first COPE polymer and no more than 30% wt/wt of a second COPE polymer. In another embodiment, the polymer composition comprises at least 80% wt/wt COPE polymer and no more than 20% wt/wt of a second COPE polymer. In another embodiment, the polymer composition comprises at least 90% wt/wt of a first COPE polymer and no more than 10% wt/wt of a second COPE polymer. In another embodiment, the polymer composition comprises at least 40% wt/wt of a first COPE polymer and no more than 60% wt/wt of a second COPE polymer. In another embodiment, the polymer composition comprises at least 30% wt/wt of a first COPE polymer and no more than 70% wt/wt of a second COPE polymer. In another embodiment, the polymer composition comprises at least 20% wt/wt of a first COPE polymer and no more than 80% wt/wt of a second COPE polymer. In another embodiment, the polymer composition comprises at least 10% wt/wt of a first COPE polymer and no more than 90% wt/wt of a second COPE polymer.

Referring now to FIG. 2, a polymeric material 208 corresponding to the polymeric material 108 is provided. The polymeric material 208 is provided in the form of an elongated cylindrical body 209 defined by a sidewall 210 extending from a first end 212 to a second end 214. A channel 211 may extend entirely through the sidewall 210 from the first end 212 and to the second end 214. The sidewall 210 may be provided in a variety of thicknesses, and the channel 211 may be provided in a variety of diameters. When the polymeric material 208 is provided as part of an apparatus (e.g., the apparatus 100 of FIG. 1) a water-containing fluid may flow through the channel 211 in a direction represented by an arrow 215. In other embodiments, the water-containing fluid may flow in other directions through the channel 211 (e.g., in a direction opposite the direction 215).

In addition, while the body 209 of the polymeric material 208 has been described as an elongated cylinder, one skilled in the art would appreciate that the body 209 may take on a virtually unlimited number of shapes and forms. In some embodiments, the polymeric material 208 may alternatively be shaped in the form of a tube. In other embodiments, the polymeric material 208 may be fibrous, and provided in the form of a fibrous tube, a hollow fiber tube, or in an alternative fibrous shape. As would be appreciated by those skilled in the art, the polymeric material may be provided in other shapes than those described herein.

In some embodiments, the described novel polymeric material 208 shape (e.g., a hollow fiber tube) provides a way of significantly reducing and removing moisture that is superior to other fluid dissipation or moisture removal devices previously known in the art.

In yet other embodiments, the physical properties of the disclosed polymeric materials 208 allow for easier manufacture of the sampling tube compared to conventional tube materials. The disclosed polymeric materials are capable of forming a smooth inner surface of the channel 211, which allows for efficient and undistorted gas flow through the polymeric material 208.

In some embodiments, in order to provide additional strength and puncture protection, an additional outer cover structure (not illustrated) can be added to the body 209 of the polymeric material 208. In various embodiments the body 209 of the polymeric material 208 may be encased (e.g., with the outer cover structure) so that dry air can be supplied to the environment surrounding the body 209 to help improve the removal of the water vapor from water-containing fluid provided to the polymeric material 208.

In a further embodiment, the polymeric material 208 may be incorporated into only a portion of the body 209, or the polymeric material 208 may be incorporated into all of the body 209. In other embodiments, the permeable portion of the body 209 may be permeable to water vapor and moisture and may include one or more layers of the polymeric material 208. In yet other embodiments, the body 209 may be provided with a portion comprising the polymeric material 208 and a portion comprising a non-water-permeable material.

In some embodiments, the apparatus in which the polymeric material 208 is provided may include a sweep gas 216 that flows over or around the polymeric material in a directly substantially opposite of the direction 215. In other embodiments, the apparatus in which the polymeric material 208 is provided includes a sweep gas 218 that flows over or around the polymeric material in substantially the same direction as the direction 215. The sweep gases 216 and 218 may remove moisture from an outside environment (not illustrated) surrounding the polymeric material 208 and transport the moisture away from the polymeric material 208. The sweep gases 216 and 218 may help make the moisture transfer process facilitated by the polymeric material 208 more efficient.

The polymeric material 208 may be comprised of a polymer composition. Some embodiments of the polymer composition include thermoplastic urethanes which have the benefit of being mechanically tough and have chemical, microbial, and hydrolytic resistance. Some embodiments of the polymer composition include thermoplastic COPE polymers such as copolyester-ester or ether polymers, which have the benefit of having good resistance to degradation and corrosion, and a wide range of hardness and mechanical properties. Prior art polymer compositions are vulnerable to water degradation, especially at high temperatures, and are brittle. The embodiments of the polymer materials in the apparatus described and described herein are flexible, strong, easy to extrude, have excellent moisture permeability, and are more durable than prior art compositions when exposed to water, heat, and humidity.

In some embodiments, the polymeric material 208 substantially maintains other desirable properties such as strength and flexibility in addition to moisture permeability. Further, in some embodiments, the polymeric material 208 may be adapted to be mechanically robust in active environments, such as in breath sampling tubes, through which the patient may exhale or inhale medications or other active ingredients.

According to some embodiments, the polymeric material 208 may be processed to produce the desired structure of the polymeric material 208 (e.g., a tube) by way of molding. In other embodiments, the molding process of the polymeric material 208 may improve the integration of the polymeric material 208 within the desired systems and/or improve the mechanical properties of the polymeric material 208. For example, at least one connector, at least one structural support element, at least one reinforcement element, and/or any other feature can be molded with or otherwise incorporated into the polymeric material 208 to provide the body 209 with enhanced properties (e.g., increased structural integrity).

In various embodiments, the polymeric material 208 is adapted to dry or dehydrate gases such as air, oxygen (O₂), oxygenated air, carbon monoxide (CO), carbon dioxide (CO₂), or any other gas. The polymeric material 208 may further be adapted to allow the flow of the gas while also maintaining the concentrations of other gas components such as carbon dioxide, oxygen, nitrogen, or any other gas.

The polymeric material 208 may be provided in the form of a moisture permeable polymer composition. In some embodiments, the polymer composition may comprise a base component of Formula I.

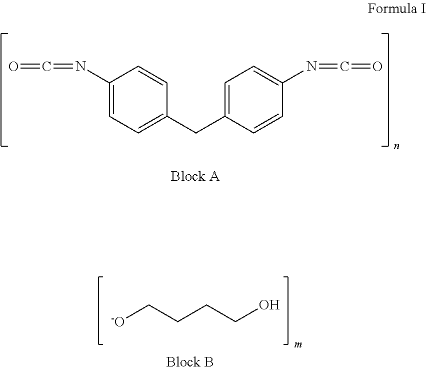

In other embodiments, the polymer composition may include two or more prepolymers, wherein the prepolymers include at least one of: (a) diisocyanate blocks connected by urethane linkages to diols of variable chain length, or (b) an alcohol or a carboxylic acid homopolymer block covalently bonded to another alcohol or carboxylic acid homopolymer.

In some embodiments, the polymer composition may comprise a block copolymer including a polyurethane block copolymer. In some embodiments, the polymer composition may be provided in the form of a polymeric material comprising a polyester polyether block copolymer (COPE). In one embodiment, the polymer composition comprises a base component of Formula II.

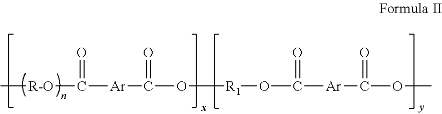

Additionally, some embodiments of the polymeric material 208 include various blends of thermoplastic urethanes (TPU) and thermoplastic polymers such as copolyester-ester or ether polymers (COPE). Some embodiments of polymeric material 208 comprise a first polymer and a second polymer, wherein the first polymer includes at least one of a polyurethane block copolymer or a polyester polyether block copolymer (COPE). In various embodiments, the TPU included in the polymeric material 208 can be an aliphatic polyether type polymer. In other embodiments, the TPU included in the polymeric material 208 can be an aliphatic blend polymer. In various embodiments of the polymeric material 208, the COPE polymer can be a polyester polyether type polymer.

The polymeric material 208 may be provided as a polymer composition including a first polymer. The first polymer may be selected from the group consisting of a polyurethane block copolymer, a polyether block copolymer, a polyester polyether block copolymer (COPE), a diisocyanate block connected by urethane linkages to diols of variable chain length, a first alcohol or a carboxylic acid homopolymer block covalently bonded to a second alcohol or carboxylic acid homopolymer, and combinations thereof. As an additional example, the polymeric material 208 may also comprise a blend of polymers including various blends of thermoplastic urethanes (TPU) and thermoplastic polymers such as copolyester-ester or ether polymers (COPE). Some embodiments of the polymeric material 108 comprise a first polymer and a second polymer, wherein the first polymer includes at least one of a polyurethane block copolymer or a polyester polyether block copolymer (COPE). In various embodiments, the TPU included in the polymeric material 208 can be an aliphatic polyether type polymer. In other embodiments, the TPU included in the polymeric material 208 can be an aliphatic blend polymer. In various embodiments of the polymeric material 208, the COPE polymer can be a polyester polyether type polymer.

In some instances, the polymeric material 208 may be provided as one or more polymers. In such instances, each polymer of the one or more polymers may be provided as any polymer described herein. By way of example, the first polymer and the second polymer may be the same, or the first polymer and the second polymer may be different.

The polymeric material 208 may be provided as a blend of polymers. For example, the blend of polymers may include at least two polymers (e.g., a first polymer and a second polymer). The first and second polymers may be any polymer that is described herein. In some instances, the first polymer may comprise the polymer blend in an amount ranging from about 0% wt/wt to about 100% wt/wt. In other instances, the second polymer may comprise the polymer blend in an amount ranging from about 0% wt/wt to about 100% wt/wt. In some instances, the first polymer may comprise the polymer blend in an amount ranging from 0% wt/wt to 100% wt/wt. In other instances, the second polymer may comprise the polymer blend in an amount ranging from 0% wt/wt to 100% wt/wt.

Yet another embodiment includes a polymer composition which comprises at least about 50% wt/wt of the first polymer and no more than about 50% wt/wt of the second polymer. In another embodiment, the polymer composition comprises at least about 60% wt/wt of the first polymer and no more than about 40% wt/wt of the second polymer. In another embodiment, the polymer composition comprises at least about 70% wt/wt of the first polymer and no more than about 30% wt/wt of the second polymer. In another embodiment, the polymer composition comprises at least about 80% wt/wt of the first polymer and no more than about 20% wt/wt of the second polymer. In another embodiment, the polymer composition comprises at least about 90% wt/wt of the first polymer and no more than about 10% wt/wt of the second polymer. In another embodiment, the polymer composition comprises at least about 40% wt/wt of the first polymer and no more than about 60% wt/wt of the second polymer. In another embodiment, the polymer composition comprises at least about 30% wt/wt of the first polymer and no more than about 70% wt/wt of the second polymer. In another embodiment, the polymer composition comprises at least about 20% wt/wt of the first polymer and no more than about 80% wt/wt of the second polymer. In another embodiment, the polymer composition comprises at least about 10% wt/wt of the first polymer and no more than about 90% wt/wt of the second polymer.

Yet another embodiment includes a polymer composition which comprises 50% wt/wt of the first polymer and 50% wt/wt of the second polymer. In another embodiment, the polymer composition comprises at least 60% wt/wt of the first polymer and no more than 40% wt/wt of the second polymer. In another embodiment, the polymer composition comprises at least 70% wt/wt of the first polymer and no more than 30% wt/wt of the second polymer. In another embodiment, the polymer composition comprises at least 80% wt/wt of the first polymer and no more than 20% wt/wt of the second polymer. In another embodiment, the polymer composition comprises at least 90% wt/wt of the first polymer and no more than 10% wt/wt of the second polymer. In another embodiment, the polymer composition comprises at least 40% wt/wt of the first polymer and no more than 60% wt/wt of the second polymer. In another embodiment, the polymer composition comprises at least 30% wt/wt of the first polymer and no more than 70% wt/wt of the second polymer. In another embodiment, the polymer composition comprises at least 20% wt/wt of the first polymer and no more than 80% wt/wt of the second polymer. In another embodiment, the polymer composition comprises at least 10% wt/wt of the first polymer and no more than 90% wt/wt of the second polymer.

Yet another embodiment includes a polymer composition which comprises about 50% wt/wt of a TPU and about 50% wt/wt of a COPE polymer. In another embodiment, the polymer composition comprises at least about 60% wt/wt of a TPU and no more than about 40% wt/wt of a COPE polymer. In another embodiment, the polymer composition comprises at least about 70% wt/wt of a TPU and no more than about 30% wt/wt of a COPE polymer. In another embodiment, the polymer composition comprises at least about 80% wt/wt of a TPU and no more than about 20% wt/wt of a COPE polymer. In another embodiment, the polymer composition comprises at least about 90% wt/wt of a TPU and no more than about 10% wt/wt of a COPE polymer. In another embodiment, the polymer composition comprises at least about 40% wt/wt of a TPU and no more than about 60% wt/wt of a COPE polymer. In another embodiment, the polymer composition comprises at least about 30% wt/wt of a TPU and no more than about 70% wt/wt of a COPE polymer. In another embodiment, the polymer composition comprises at least about 20% wt/wt of a TPU and no more than about 80% wt/wt of a COPE polymer. In another embodiment, the polymer composition comprises at least about 10% wt/wt of a TPU and no more than about 90% wt/wt of a COPE polymer.

Yet another embodiment includes a polymer composition which comprises 50% wt/wt of a TPU and 50% wt/wt of a COPE polymer. In another embodiment, the polymer composition comprises at least 60% wt/wt of a TPU and no more than 40% wt/wt of a COPE polymer. In another embodiment, the polymer composition comprises at least 70% wt/wt of a TPU and no more than 30% wt/wt of a COPE polymer. In another embodiment, the polymer composition comprises at least 80% wt/wt of a TPU and no more than 20% wt/wt of a COPE polymer. In another embodiment, the polymer composition comprises at least 90% wt/wt of a TPU and no more than 10% wt/wt of a COPE polymer. In another embodiment, the polymer composition comprises at least 40% wt/wt of a TPU and no more than 60% wt/wt of a COPE polymer. In another embodiment, the polymer composition comprises at least 30% wt/wt of a TPU and no more than 70% wt/wt of a COPE polymer. In another embodiment, the polymer composition comprises at least 20% wt/wt of a TPU and no more than 80% wt/wt of a COPE polymer. In another embodiment, the polymer composition comprises at least 10% wt/wt of a TPU and no more than 90% wt/wt of a COPE polymer.

Some embodiments include a polymer composition which comprises about 50% wt/wt of a first TPU and about 50% wt/wt of a second TPU. In another embodiment, the polymer composition comprises at least about 70% wt/wt of a first TPU and no more than about 30% wt/wt of a second TPU polymer. In another embodiment, the polymer composition comprises at least about 80% wt/wt TPU and no more than about 20% wt/wt of a second TPU. In another embodiment, the polymer composition comprises at least about 90% wt/wt of a first TPU and no more than about 10% wt/wt of a second TPU. In another embodiment, the polymer composition comprises at least about 40% wt/wt of a first TPU and no more than about 60% wt/wt of a second TPU. In another embodiment, the polymer composition comprises at least about 30% wt/wt of a first TPU and no more than about 70% wt/wt of a second TPU. In another embodiment, the polymer composition comprises at least about 20% wt/wt of a first TPU and no more than about 80% wt/wt of a second TPU. In another embodiment, the polymer composition comprises at least about 10% wt/wt of a first TPU and no more than about 90% wt/wt of a second TPU.

Some embodiments include a polymer composition which comprises 50% wt/wt of a first TPU and 50% wt/wt of a second TPU. In another embodiment, the polymer composition comprises at least 70% wt/wt of a first TPU and no more than 30% wt/wt of a second TPU polymer. In another embodiment, the polymer composition comprises at least 80% wt/wt TPU and no more than 20% wt/wt of a second TPU. In another embodiment, the polymer composition comprises at least 90% wt/wt of a first TPU and no more than 10% wt/wt of a second TPU. In another embodiment, the polymer composition comprises at least 40% wt/wt of a first TPU and no more than 60% wt/wt of a second TPU. In another embodiment, the polymer composition comprises at least 30% wt/wt of a first TPU and no more than 70% wt/wt of a second TPU. In another embodiment, the polymer composition comprises at least 20% wt/wt of a first TPU and no more than 80% wt/wt of a second TPU. In another embodiment, the polymer composition comprises at least 10% wt/wt of a first TPU and no more than 90% wt/wt of a second TPU.

Some embodiments include a polymer composition which comprises about 50% wt/wt of a first COPE polymer and about 50% wt/wt of a second COPE polymer. In another embodiment, the polymer composition comprises at least about 70% wt/wt of a first COPE polymer and no more than about 30% wt/wt of a second COPE polymer. In another embodiment, the polymer composition comprises at least about 80% wt/wt COPE polymer and no more than about 20% wt/wt of a second COPE polymer. In another embodiment, the polymer composition comprises at least about 90% wt/wt of a first COPE polymer and no more than about 10% wt/wt of a second COPE polymer. In another embodiment, the polymer composition comprises at least about 40% wt/wt of a first COPE polymer and no more than about 60% wt/wt of a second COPE polymer. In another embodiment, the polymer composition comprises at least about 30% wt/wt of a first COPE polymer and no more than about 70% wt/wt of a second COPE polymer. In another embodiment, the polymer composition comprises at least about 20% wt/wt of a first COPE polymer and no more than about 80% wt/wt of a second COPE polymer. In another embodiment, the polymer composition comprises at least about 10% wt/wt of a first COPE polymer and no more than about 90% wt/wt of a second COPE polymer.

Some embodiments include a polymer composition which comprises 50% wt/wt of a first COPE polymer and 50% wt/wt of a second COPE polymer. In another embodiment, the polymer composition comprises at least 70% wt/wt of a first COPE polymer and no more than 30% wt/wt of a second COPE polymer. In another embodiment, the polymer composition comprises at least 80% wt/wt COPE polymer and no more than 20% wt/wt of a second COPE polymer. In another embodiment, the polymer composition comprises at least 90% wt/wt of a first COPE polymer and no more than 10% wt/wt of a second COPE polymer. In another embodiment, the polymer composition comprises at least 40% wt/wt of a first COPE polymer and no more than 60% wt/wt of a second COPE polymer. In another embodiment, the polymer composition comprises at least 30% wt/wt of a first COPE polymer and no more than 70% wt/wt of a second COPE polymer. In another embodiment, the polymer composition comprises at least 20% wt/wt of a first COPE polymer and no more than 80% wt/wt of a second COPE polymer. In another embodiment, the polymer composition comprises at least 10% wt/wt of a first COPE polymer and no more than 90% wt/wt of a second COPE polymer.

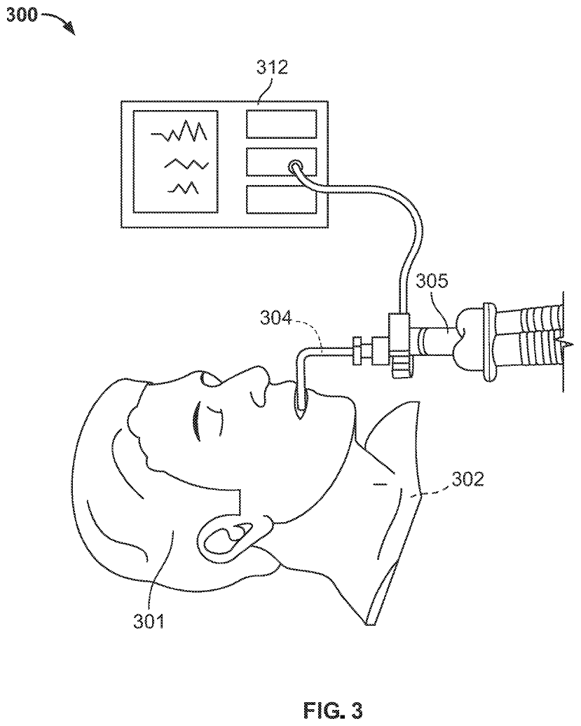

Turning now to FIG. 3, a respiratory apparatus 300 is designed to be used with a patient or a subject 301 and to analyze a breath source 302. A breath gas 304 is released from the subject 301 and passed through to a respiratory interface 305 and an analyzer 312. The apparatus 300 may have a substantially similar structure and function as the apparatus 100. Further, similarly named and/or numbered components of the apparatus 300 may have substantially the same function and configuration as the similarly named and/or numbered components described with reference to FIGS. 1 and 2.

The breath source 302 may be provided from lungs (not illustrated) of the subject 301. For example, the breath source 302 may be generated from the respiratory processes carried out in the lungs of the subject 301. While the subject 301 is illustrated as a human, the subject 301 may be any animal or human.

The breath gas 304 may be provided as exhaled air from the breath source 302. The breath gas 304 may be imparted with a first level of moisture or concentration of water, as well as exhaled gases (e.g., nitrogen (N₂) and carbon dioxide (CO₂)).

The respiratory interface 305 may be an intubation device, a nasal prong, a nasal cannula, an oral prong, a conical fitting, and/or other similar devices known in the art. The gas analyzer 312 may be configured to measure the concentration of oxygen, carbon dioxide, carbon monoxide, and/or nitrogen in the breath gas 304.

The apparatus 300 may be designed to remove moisture from the breath gases 304 of the subject 301. Medical breath analysis often requires that the sample gas be free or substantially free of liquid. One example of gas analysis is capnography, which monitors respiratory concentration of gases over time. In various embodiments, the apparatus can be applied to capnography systems. In other embodiments, the apparatus 300 may include gas analysis systems measuring the concentration of oxygen, carbon dioxide, carbon monoxide, and nitrogen in respiratory gases, individually or in combination.

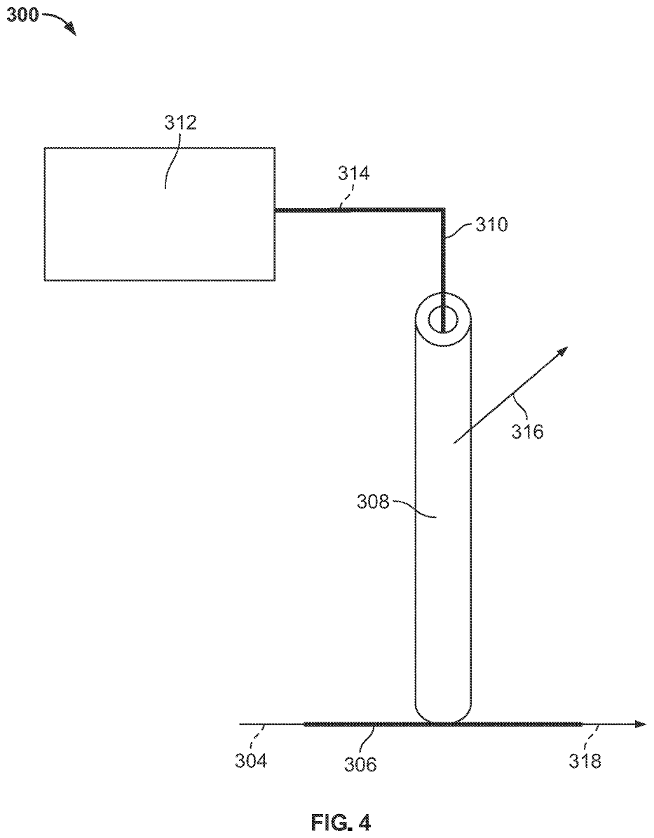

Referring now to FIG. 4, the apparatus 300 is schematically illustrated such that a first conduit 306, a polymeric material 308, a second conduit 310, the analyzer 312, a dehydrated gas 314, water 316, and non-sampled gases 318 are visible. As stated previously, similarly named and/or numbered components of the apparatus 300 may have substantially the same function and configuration as the similarly named and/or numbered components described with reference to FIGS. 1 and 2. Like the apparatus 100 illustrated in FIG. 1, the apparatus 300 may remove moisture from a fluid (here, the breath gas 304) using a polymeric material (here, the polymeric material 308). More specifically, the polymeric material 308 is adapted to remove water 316 from the breath gas 304 to create the dehydrated gas 314, which is transferred through the second conduit 310. The second conduit 310 may be in fluid communication with an outlet (not illustrated). Alternatively, or additionally, the dehydrated gas 314 may be transferred through the second conduit 310 to the analyzer 312. Additionally, the analyzer 312 is configured to measure at least one parameter of the dehydrated gas.

The analyzer 312 may be any analyzer capable of measuring analytes in the sample including but not limited to oxygen, carbon dioxide, carbon monoxide, and nitrogen. The analyzer 312 may be provided in the form of an infrared spectrometer, a UV-Vis spectrometer, a Raman spectrometer, gas chromatography system, or a mass spectrometer. In various embodiments, the analyzer 312 may be configured to measure the concentration of oxygen, carbon dioxide, carbon monoxide, and nitrogen in the patient's breath gas 304.

In some embodiments, the analyzer 312 may be provided as a sidestream gas analyzer such that non-sampled gases 318 may continue traveling through the respiratory interface 305. In other embodiments, the provided analyzer 312 is a mainstream gas analyzer. Furthermore, the various embodiments have further advantages as the apparatus 300 may remove moisture from the gases or liquids without any significant loss of the desired analytes. Additionally, in some embodiments, the removal of moisture from the breath gas 304 provides improved analytical outcomes and protects equipment from degradation. In some embodiments, the apparatus 300 is designed to continuously sample the patient 301 breath and provide the breath gas 304 in a tube that is in fluid communication with the analyzer 312 while drying out the breath gas 304 via the polymeric material 308.

In some embodiments, the polymeric material 308 of the apparatus 300 may be provided as described in FIGS. 1 and 2. In some embodiments, the polymeric material 308 may shaped as an elongated cylinder, or alternatively be shaped in the form of a tube (e.g., see FIG. 2).

The polymeric material 308 may be provided in the form of a moisture permeable polymer composition. In some embodiments, the polymer composition may comprise a base component of Formula I

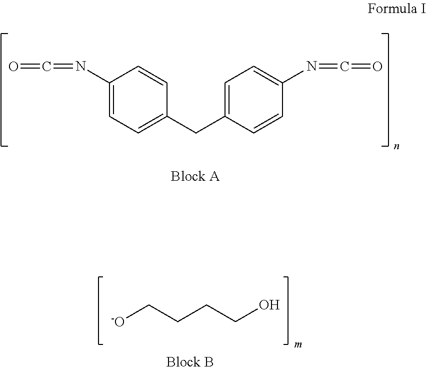

In other embodiments, the polymer composition may include two or more prepolymers, wherein the prepolymers include at least one of: (a) diisocyanate blocks connected by urethane linkages to diols of variable chain length or (b) an alcohol or a carboxylic acid homopolymer block covalently bonded to another alcohol or carboxylic acid homopolymer.

In some embodiments, the polymer composition may comprise a block copolymer including a polyurethane block copolymer. In some embodiments, the polymer composition may be in the form of a polymeric material comprising a polyester polyether block copolymer (COPE). In one embodiment, the polymer composition comprises a base component of Formula II.

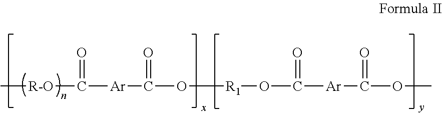

Additionally, some embodiments of the polymeric material 308 include various blends of thermoplastic urethanes (TPU) and thermoplastic polymers such as copolyester-ester or ether polymers (COPE). Some embodiments of polymeric material 308 comprise a first polymer and a second polymer, wherein the first polymer includes at least one of a polyurethane block copolymer or a polyester polyether block copolymer (COPE). In various embodiments, the TPU included in the polymeric material 308 can be an aliphatic polyether type polymer. In other embodiments, the TPU included in the polymeric material 308 can be an aliphatic blend polymer. In various embodiments of the polymeric material 308, the COPE polymer can be a polyester polyether type polymer.

Additionally, some embodiments of the polymeric material 308 include various blends of thermoplastic urethanes (TPU) and thermoplastic polymers such as copolyester-ester or ether polymers (COPE). Some embodiments of polymeric material 308 comprise a first polymer and a second polymer, wherein the first polymer includes at least one of a polyurethane block copolymer or a polyester polyether block copolymer (COPE). In various embodiments, the TPU included in the polymeric material 308 can be an aliphatic polyether type polymer. In other embodiments, the TPU included in the polymeric material 308 can be an aliphatic blend polymer. In various embodiments of the polymeric material 308, the COPE polymer can be a polyester polyether type polymer.

The polymeric material 308 may be provided as a polymer composition including a first polymer. The first polymer may be selected from the group consisting of a polyurethane block copolymer, a polyether block copolymer, a polyester polyether block copolymer (COPE), a diisocyanate block connected by urethane linkages to diols of variable chain length, a first alcohol or a carboxylic acid homopolymer block covalently bonded to a second alcohol or carboxylic acid homopolymer, and combinations thereof. As an additional example the polymeric material 308 may also comprise a blend of polymers including various blends of thermoplastic urethanes (TPU) and thermoplastic polymers such as copolyester-ester or ether polymers (COPE). Some embodiments of polymeric material 308 comprise a first polymer and a second polymer, wherein the first polymer includes at least one of a polyurethane block copolymer or a polyester polyether block copolymer (COPE). In various embodiments, the TPU included in the polymeric material 108 can be an aliphatic polyether type polymer. In other embodiments, the TPU included in the polymeric material 308 can be an aliphatic blend polymer. In various embodiments of the polymeric material 308, the COPE polymer can be a polyester polyether type polymer.

In some instances, the polymeric material 308 may be provided as one or more polymers. In such instances, each polymer of the one or more polymers may be provided as any polymer described herein.

The polymeric material 308 may be provided as a blend of polymers. For example, the blend of polymers may include at least two polymers (e.g., a first polymer and a second polymer). The first and second polymers may be any polymer that is described herein. In some instances, the first polymer may comprise the polymer blend in an amount ranging from about 0% wt/wt to about 100% wt/wt. In other instances, the second polymer may comprise the polymer blend in an amount ranging from about 0% wt/wt to about 100% wt/wt. In some instances, the first polymer may comprise the polymer blend in an amount ranging from 0% wt/wt to 100% wt/wt. In other instances, the second polymer may comprise the polymer blend in an amount ranging from 0% wt/wt to 100% wt/wt.

Yet another embodiment includes a polymer composition which comprises at least about 50% wt/wt of the first polymer and no more than about 50% wt/wt of the second polymer. In another embodiment, the polymer composition comprises at least about 60% wt/wt of the first polymer and no more than about 40% wt/wt of the second polymer. In another embodiment, the polymer composition comprises at least about 70% wt/wt of the first polymer and no more than about 30% wt/wt of the second polymer. In another embodiment, the polymer composition comprises at least about 80% wt/wt of the first polymer and no more than about 20% wt/wt of the second polymer. In another embodiment, the polymer composition comprises at least about 90% wt/wt of the first polymer and no more than about 10% wt/wt of the second polymer. In another embodiment, the polymer composition comprises at least about 40% wt/wt of the first polymer and no more than about 60% wt/wt of the second polymer. In another embodiment, the polymer composition comprises at least about 30% wt/wt of the first polymer and no more than about 70% wt/wt of the second polymer. In another embodiment, the polymer composition comprises at least about 20% wt/wt of the first polymer and no more than about 80% wt/wt of the second polymer. In another embodiment, the polymer composition comprises at least about 10% wt/wt of the first polymer and no more than about 90% wt/wt of the second polymer.

Yet another embodiment includes a polymer composition which comprises 50% wt/wt of the first polymer and 50% wt/wt of the second polymer. In another embodiment, the polymer composition comprises at least 60% wt/wt of the first polymer and no more than 40% wt/wt of the second polymer. In another embodiment, the polymer composition comprises at least 70% wt/wt of the first polymer and no more than 30% wt/wt of the second polymer. In another embodiment, the polymer composition comprises at least 80% wt/wt of the first polymer and no more than 20% wt/wt of the second polymer. In another embodiment, the polymer composition comprises at least 90% wt/wt of the first polymer and no more than 10% wt/wt of the second polymer. In another embodiment, the polymer composition comprises at least 40% wt/wt of the first polymer and no more than 60% wt/wt of the second polymer. In another embodiment, the polymer composition comprises at least 30% wt/wt of the first polymer and no more than 70% wt/wt of the second polymer. In another embodiment, the polymer composition comprises at least 20% wt/wt of the first polymer and no more than 80% wt/wt of the second polymer. In another embodiment, the polymer composition comprises at least 10% wt/wt of the first polymer and no more than 90% wt/wt of the second polymer.

Yet another embodiment includes a polymer composition which comprises about 50% wt/wt of a TPU and about 50% wt/wt of a COPE polymer. In another embodiment, the polymer composition comprises at least about 60% wt/wt of a TPU and no more than about 40% wt/wt of a COPE polymer. In another embodiment, the polymer composition comprises at least about 70% wt/wt of a TPU and no more than about 30% wt/wt of a COPE polymer. In another embodiment, the polymer composition comprises at least about 80% wt/wt of a TPU and no more than about 20% wt/wt of a COPE polymer. In another embodiment, the polymer composition comprises at least about 90% wt/wt of a TPU and no more than about 10% wt/wt of a COPE polymer. In another embodiment, the polymer composition comprises at least about 40% wt/wt of a TPU and no more than about 60% wt/wt of a COPE polymer. In another embodiment, the polymer composition comprises at least about 30% wt/wt of a TPU and no more than about 70% wt/wt of a COPE polymer. In another embodiment, the polymer composition comprises at least about 20% wt/wt of a TPU and no more than about 80% wt/wt of a COPE polymer. In another embodiment, the polymer composition comprises at least about 10% wt/wt of a TPU and no more than about 90% wt/wt of a COPE polymer.

Yet another embodiment includes a polymer composition which comprises 50% wt/wt of a TPU and 50% wt/wt of a COPE polymer. In another embodiment, the polymer composition comprises at least 60% wt/wt of a TPU and no more than 40% wt/wt of a COPE polymer. In another embodiment, the polymer composition comprises at least 70% wt/wt of a TPU and no more than 30% wt/wt of a COPE polymer. In another embodiment, the polymer composition comprises at least 80% wt/wt of a TPU and no more than 20% wt/wt of a COPE polymer. In another embodiment, the polymer composition comprises at least 90% wt/wt of a TPU and no more than 10% wt/wt of a COPE polymer. In another embodiment, the polymer composition comprises at least 40% wt/wt of a TPU and no more than 60% wt/wt of a COPE polymer. In another embodiment, the polymer composition comprises at least 30% wt/wt of a TPU and no more than 70% wt/wt of a COPE polymer. In another embodiment, the polymer composition comprises at least 20% wt/wt of a TPU and no more than 80% wt/wt of a COPE polymer. In another embodiment, the polymer composition comprises at least 10% wt/wt of a TPU and no more than 90% wt/wt of a COPE polymer.

Some embodiments include a polymer composition which comprises about 50% wt/wt of a first TPU and about 50% wt/wt of a second TPU. In another embodiment, the polymer composition comprises at least about 70% wt/wt of a first TPU and no more than about 30% wt/wt of a second TPU polymer. In another embodiment, the polymer composition comprises at least about 80% wt/wt TPU and no more than about 20% wt/wt of a second TPU. In another embodiment, the polymer composition comprises at least about 90% wt/wt of a first TPU and no more than about 10% wt/wt of a second TPU. In another embodiment, the polymer composition comprises at least about 40% wt/wt of a first TPU and no more than about 60% wt/wt of a second TPU. In another embodiment, the polymer composition comprises at least about 30% wt/wt of a first TPU and no more than about 70% wt/wt of a second TPU. In another embodiment, the polymer composition comprises at least about 20% wt/wt of a first TPU and no more than about 80% wt/wt of a second TPU. In another embodiment, the polymer composition comprises at least about 10% wt/wt of a first TPU and no more than about 90% wt/wt of a second TPU.

Some embodiments include a polymer composition which comprises 50% wt/wt of a first TPU and 50% wt/wt of a second TPU. In another embodiment, the polymer composition comprises at least 70% wt/wt of a first TPU and no more than 30% wt/wt of a second TPU polymer. In another embodiment, the polymer composition comprises at least 80% wt/wt TPU and no more than 20% wt/wt of a second TPU. In another embodiment, the polymer composition comprises at least 90% wt/wt of a first TPU and no more than 10% wt/wt of a second TPU. In another embodiment, the polymer composition comprises at least 40% wt/wt of a first TPU and no more than 60% wt/wt of a second TPU. In another embodiment, the polymer composition comprises at least 30% wt/wt of a first TPU and no more than 70% wt/wt of a second TPU. In another embodiment, the polymer composition comprises at least 20% wt/wt of a first TPU and no more than 80% wt/wt of a second TPU. In another embodiment, the polymer composition comprises at least 10% wt/wt of a first TPU and no more than 90% wt/wt of a second TPU.

Some embodiments include a polymer composition which comprises about 50% wt/wt of a first COPE polymer and about 50% wt/wt of a second COPE polymer. In another embodiment, the polymer composition comprises at least about 70% wt/wt of a first COPE polymer and no more than about 30% wt/wt of a second COPE polymer. In another embodiment, the polymer composition comprises at least about 80% wt/wt COPE polymer and no more than about 20% wt/wt of a second COPE polymer. In another embodiment, the polymer composition comprises at least about 90% wt/wt of a first COPE polymer and no more than about 10% wt/wt of a second COPE polymer. In another embodiment, the polymer composition comprises at least about 40% wt/wt of a first COPE polymer and no more than about 60% wt/wt of a second COPE polymer. In another embodiment, the polymer composition comprises at least about 30% wt/wt of a first COPE polymer and no more than about 70% wt/wt of a second COPE polymer. In another embodiment, the polymer composition comprises at least about 20% wt/wt of a first COPE polymer and no more than about 80% wt/wt of a second COPE polymer. In another embodiment, the polymer composition comprises at least about 10% wt/wt of a first COPE polymer and no more than about 90% wt/wt of a second COPE polymer.

Some embodiments include a polymer composition which comprises 50% wt/wt of a first COPE polymer and 50% wt/wt of a second COPE polymer. In another embodiment, the polymer composition comprises at least 70% wt/wt of a first COPE polymer and no more than 30% wt/wt of a second COPE polymer. In another embodiment, the polymer composition comprises at least 80% wt/wt COPE polymer and no more than 20% wt/wt of a second COPE polymer. In another embodiment, the polymer composition comprises at least 90% wt/wt of a first COPE polymer and no more than 10% wt/wt of a second COPE polymer. In another embodiment, the polymer composition comprises at least 40% wt/wt of a first COPE polymer and no more than 60% wt/wt of a second COPE polymer. In another embodiment, the polymer composition comprises at least 30% wt/wt of a first COPE polymer and no more than 70% wt/wt of a second COPE polymer. In another embodiment, the polymer composition comprises at least 20% wt/wt of a first COPE polymer and no more than 80% wt/wt of a second COPE polymer. In another embodiment, the polymer composition comprises at least 10% wt/wt of a first COPE polymer and no more than 90% wt/wt of a second COPE polymer.

In another embodiment, the apparatus of FIG. 1 may be used to remove dissolved and free water from lubrication and hydraulic fluids using a polymeric material (e.g., the polymeric materials 108, 208, and/or 308). The lubrication and hydraulic fluids may be largely unable to pass through the polymeric tubing, but moisture may do so easily. In some embodiments, the moisture may be removed on the other side of the polymeric material by a dry sweep gas, by vacuum, or by ambient air contact.

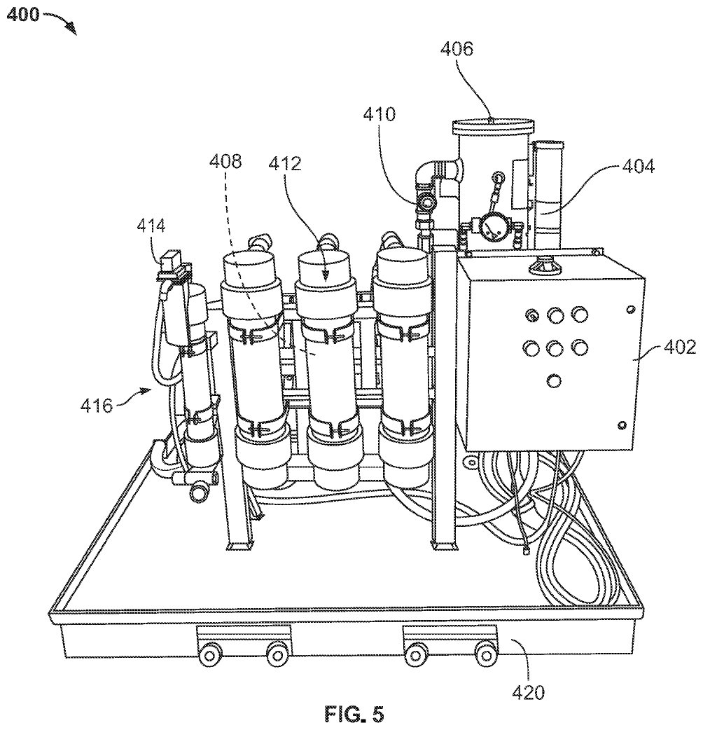

Referring now to FIG. 5, an oil conditioning system 400 is provided in the form of a control panel 402, a beacon light 404, a prefilter 406, a filter indicator 410, a vessel 412 retaining a polymeric material 408, an inlet air coalescer 414, an air dryer 416, and a mounting frame 420. The system 400 may be designed to filter and remove solid contaminants and water from the oils and/or other fluids (not illustrated) used within lubrication and hydraulic systems. Optionally, the output of the system 400 may be provided to an analyzer (not illustrated) designed to measure at least one parameter of the fluids conditioned with and/or passing through the system 400.

The control panel 402 may be designed to enable a user to change various computer hardware and software functions of the system 400. In some instances, the control panel 402 may include a protective shutdown mechanism. For example, the shutdown mechanism may be a series of components or software logic designed to sense a predetermined signal and halt an activity of the system 400 upon identification or detection of the predetermined signal. In some instances, the control panel 402 is not provided with the system 400. In such instances, the system 400 may be remotely controlled by a controller (not illustrated).

The beacon light 404 may be provided as an LED or other light-emitting structure that is configured to visually communicate information to a user. For example, the beacon light 404 may be configured to communicate information related to the run status of the system 400, and/or the beacon light 404 may act as a fault indicator. In some embodiments, the run status may be an indicator an action that the system 400 is presently executing. In some embodiments, the fault indicator may be a signal indicating that the system 400 is not functioning as designed or intended. In some instances, the beacon light 404 is omitted from the system 400.

The prefilter 406 may be provided as any filter configured to remove particulates from a fluid. For example, the prefilter 406 may be provided as a filter cartridge including a membrane or porous structure designed to filter particulates from a fluid based on the size of the particulates. The filter indicator 410 may signal to the user when the prefilter 406 or other filters provided with the system 400 need replacement. In some instances, the prefilter 406 and/or the filter indicator 410 may be omitted from the system 400.

The vessel 412 may be designed to retain the polymeric material 408. For example, the vessel 412 may be provided as a substantially hollow, cylindrical structure that is in fluid communication with the other components of the system 400. In addition, the vessel 412 (and the polymeric material 408 within) may be designed to remove water from the oils or fluids provided to the system 400. For example, after passing through the vessel 412, the water content of the oil may decrease to less than about 100 ppm. As an additional example, after passing through the vessel 412, the water content of the oil may decrease to less than about 100 ppm, or less than about 90 ppm, or less than about 80 ppm, or less than about 70 ppm, or less than about 60 ppm, or less than about 50 ppm, or less than about 40 ppm, or less than about 30 ppm, or less than about 20 ppm.

In some embodiments, the system 400 may include the inlet air coalescer 414 and the air dryer 416 to further process the oil-based fluid and/or compressed air provided to the system 400. For example, the inlet air coalescer 414 may be configured to remove oil, water, or particulates from compressed air provided to the system 400. As an additional example, the air dryer 416 may be designed for oil dehydration and may comprise heated air and/or a heating element.

In some embodiments, the mounting frame 420 provides a structure to which the other components of the system 400 can be coupled thereto. In addition, the mounting frame 420 may be forklift and pallet-jack couplable to help facilitate easy transport of the system 400.

The vessel 412 may include the polymeric material 408. The polymeric material 408 may be provided with the same structure and compositions as the polymeric materials 108, 208, and 308, or the polymeric material 408 may have a different structure or composition than the polymeric materials 108, 208, and 308. For example, the polymeric material 408 may be provided as a polymer composition including a first polymer. The first polymer may be selected from the group consisting of a polyurethane block copolymer, a polyether block copolymer, a polyester polyether block copolymer (COPE), a diisocyanate block connected by urethane linkages to diols of variable chain length, a first alcohol or a carboxylic acid homopolymer block covalently bonded to a second alcohol or carboxylic acid homopolymer, and combinations thereof. As an additional example the polymeric material 408 may also comprise a blend of polymers including various blends of thermoplastic urethanes (TPU) and thermoplastic polymers such as copolyester-ester or ether polymers (COPE). Some embodiments of polymeric material 408 comprise a first polymer and a second polymer, wherein the first polymer includes at least one of a polyurethane block copolymer or a polyester polyether block copolymer (COPE). In various embodiments, the TPU included in the polymeric material 408 can be an aliphatic polyether type polymer. In other embodiments, the TPU included in the polymeric material 408 can be an aliphatic blend polymer. In various embodiments of the polymeric material 408, the COPE polymer can be a polyester polyether type polymer. Other embodiments of polymeric material 408 comprising blends of a first and second polymer are substantially the same as in polymeric material 208 (see FIG. 2).

Methods for removing water content or moisture content from a fluid are also provided. In some instances, the methods provided herein may include the use of a moisture removal apparatus, such as the apparatus 100, 300, and 400 described herein. The methods may utilize any embodiment of the apparatuses that are consistent with the teachings recited herein. In addition, the methods may include any of the polymeric materials 108, 208, 308, and 408, and any variations thereof, described herein.

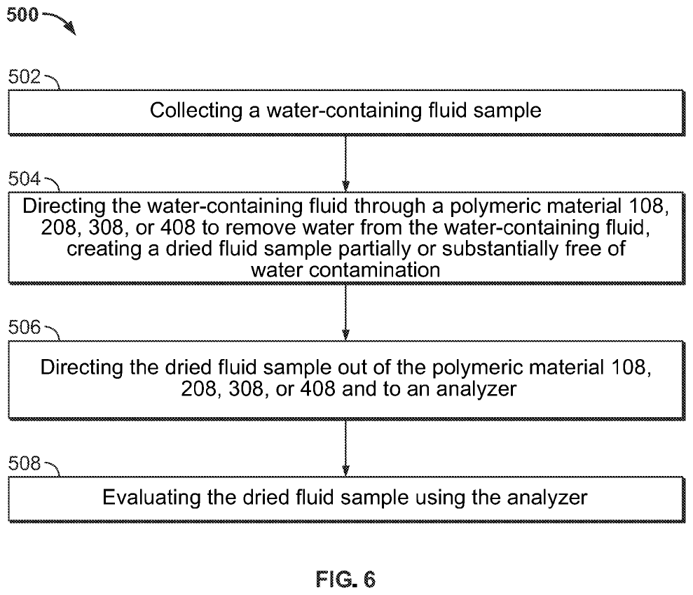

Referring now to FIG. 6, a method 500 for removing moisture from a fluid is provided. The method 500 may include a step 502 of collecting a water-containing fluid sample. The method 500 may also include a step 504 of directing the water-containing fluid through a polymeric material 108, 208, 308, or 408 to remove water from the water-containing fluid, thereby creating a dehydrated fluid sample partially or substantially free of water contamination. The method 500 may also include a step 506 of directing the dehydrated fluid sample out of the polymeric material 108, 208, 308, or 408 and to an analyzer. The method 500 may further include a step 508 of evaluating the dehydrated fluid sample using the analyzer.

The polymeric material utilized in the step 504 may be provided in various forms. For example, the polymeric material may be provided in the form of a tube. As an additional example, the polymeric material may be provided in the form of a body shaped as an elongated cylinder. A sidewall of the body may define a channel that extends through the body. As yet another example, the polymeric material may be fibrous, in the form of a fibrous tube, in the form of a hollow fiber tube, or provided in an alternative fibrous shape.

In some embodiments of the step 504, the dehydrated fluid sample is partially free of water contamination. For example, the dehydrated fluid sample may have at least about 10% less, or at least about 20% less, or at least about 30% less, or at least 40% less, or at least about 50% less moisture, or at least about 60% less moisture than the water-containing fluid. In other embodiments of the step 504, the dehydrated fluid sample is substantially free of water contamination. For example, the dehydrated fluid sample may have at least about 50% less, or at least about 60% less, or at least about 70% less, or at least about 80% less, or at least 90% less, or at least about 100% less moisture than the water-containing fluid.

Some embodiments of the method 500 further include a step wherein the polymeric material is provided as a polymer composition including a first polymer. The first polymer is selected from the group consisting of a polyurethane block copolymer, a polyether block copolymer, a polyester polyether block copolymer (COPE), a diisocyanate block connected by urethane linkages to diols of variable chain length, a first alcohol or a carboxylic acid homopolymer block covalently bonded to a second alcohol or carboxylic acid homopolymer, and combinations thereof.

Other embodiments of the method 500 also include a step of using an analyzer that is configured to measure at least one parameter of the dehydrated fluid sample. Optionally, the analyzer may be provided as a gas analyzer. The gas analyzer may be an infrared spectrometer, a UV-Vis spectrometer, a Raman spectrometer, gas chromatography system, or a mass spectrometer. In various embodiments, the gas analyzer may be configured to measure the concentration of oxygen, carbon dioxide, carbon monoxide, and nitrogen in the dehydrated fluid sample.

In some instances, the water-containing fluid sample may be imparted with a first concentration of water and the dehydrated fluid sample may be imparted with a second concentration of water. In such instances, the first concentration of water may be greater than the second concentration of water.

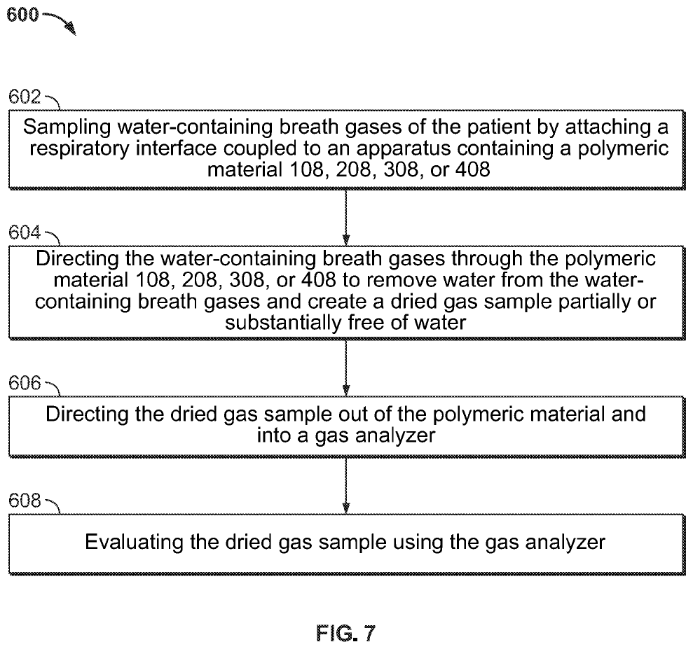

Referring now to FIG. 7, a method 600 for removing moisture from a breath gas is provided. The method 600 may include a step 602 of sampling water-containing breath gases of a patient by attaching a respiratory interface coupled to an apparatus containing a polymeric material 108, 208, 308, or 408. The method 600 may also include a step 604 of directing the water-containing breath gases through the polymeric material 108, 208, 308, or 408 to remove water from the water-containing breath gases and creating a dehydrated gas sample partially or substantially free of water. The method may include a step 606 of directing the dehydrated gas sample out of the polymeric material and into a gas analyzer. The method may additionally include a step 608 of evaluating the dehydrated gas sample using the gas analyzer.

The polymeric material utilized in the step 604 may be provided in various forms. For example, the polymeric material may be provided in the form of a tube. As an additional example, the polymeric material may be provided in the form of a body shaped as an elongated cylinder. A sidewall of the body may define a channel that extends through the body. As yet another example, the polymeric material may be fibrous, in the form of a fibrous tube, in the form of a hollow fiber tube, or provided in an alternative fibrous shape.

In some embodiments of the step 604, the dehydrated fluid sample is partially free of water contamination. For example, the dehydrated fluid sample may have at least about 10% less, or at least about 20% less, or at least about 30% less, or at least 40% less, or at least about 50% less moisture, or at least about 60% less moisture than the water-containing fluid. In other embodiments of the step 604, the dehydrated fluid sample is substantially free of water contamination. For example, the dehydrated fluid sample may have at least about 60% less, or at least about 70% less, or at least about 80% less, or at least 90% less, or at least about 100% less moisture than the water-containing fluid.

Some embodiments of the method 600 further include a step wherein the polymeric material is provided as a polymer composition including a first polymer. The first polymer is selected from the group consisting of a polyurethane block copolymer, a polyether block copolymer, a polyester polyether block copolymer (COPE), a diisocyanate block connected by urethane linkages to diols of variable chain length, a first alcohol or a carboxylic acid homopolymer block covalently bonded to a second alcohol or carboxylic acid homopolymer, and combinations thereof.

Other embodiments of the method 600 also include a step of using a gas analyzer that is configured to measure at least one parameter of the dehydrated gas. The gas analyzer may be an infrared spectrometer, a UV-Vis spectrometer, a Raman spectrometer, gas chromatography system, or a mass spectrometer. In various embodiments, the gas analyzer may be configured to measure the concentration of oxygen, carbon dioxide, carbon monoxide, and nitrogen in the breath gases of the patient.

In some instances, the water-containing fluid sample may be imparted with a first concentration of water and the dehydrated fluid sample may be imparted with a second concentration of water. In such instances, the first concentration of water may be greater than the second concentration of water.

The term “polymer” may include any molecule composed of repeating structural units connected to each other, typically, by covalent chemical bonds. The term “polymer” may further include a homopolymer (which is a polymer derived from one monomer species), a copolymer (which is a polymer derived from two or more monomeric species) or a combination thereof. A polymer, as referred to herein, may also include a mixture of polymers. A polymer, as referred to herein, may include linear and/or branched polymers which consist of a single main chain with one or more polymeric side chains. The term “polymer” may also refer to an oligomer or pre-polymer or any other chemical structure similar to the descriptions provided herein.

A copolymer, as referred to herein, may include an alternating copolymer, a periodic copolymer, a random copolymer, a block copolymer, or any combination thereof.

According to some embodiments of the present disclosure, a process is provided that includes the transfer of fluid(s) through a membrane (such as the walls of a polymer tube) wherein the fluid(s) enter the non-porous or porous membrane as vapor or liquid and permeate through the membrane as vapor. The fluid(s) may include water, humidity, water vapor or any other fluid.

The present disclosure offers the following technical advantages over existing solutions: improved apparatus for removing moisture from a fluid without distorting gas flow or the concentration of the sampled gases, improved molding capabilities for manufacture based on the polymeric composition, and increased competitive options to the medical practitioner and improved toughness and durability compared to the existing offerings in the market.

It will be appreciated by those skilled in the art that while the disclosure has been described above in connection with particular embodiments and examples, the disclosure is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the disclosure are set forth in the following claims.

1. A fluid dehydrating apparatus, comprising:
a source of a fluid, the fluid imparted with a first concentration of water;
a first conduit in fluid communication with the source of the fluid;
a polymeric material defined by a cylindrical sidewall having a channel extending therethrough, and wherein the polymeric material receives the fluid from the first conduit and removes water from the fluid to create a dehydrated fluid imparted with a second concentration of water that is less than the first concentration of water; and
a second conduit coupled to the second end of the polymeric material, the second conduit in fluid communication with an outlet.

2. The apparatus of claim 1 further including an analyzer in fluid communication with the second conduit and designed to measure at least one parameter of the dehydrated fluid.

3. The apparatus of claim 1 further including a sweep gas that flows over the polymeric material, wherein the sweep gas transfers water away from the polymeric material.

4. The apparatus of claim 1, wherein the fluid is selected from the group consisting of a breath gas, a hydraulic fluid, a lubrication oil, a transformer oil, or a liquid fuel.

5. The apparatus of claim 1, wherein the polymeric material comprises a block copolymer including a polyurethane block copolymer.

6. The apparatus of claim 1, wherein the polymeric material comprises at least one of a block copolymer including a polyether or a polyester polyether block copolymer (COPE).

7. The apparatus of claim 1, wherein the polymeric material comprises a first polymer that includes at least one of a polyurethane block copolymer or a polyester polyether block copolymer (COPE), and a second polymer.

8. A fluid dehydrating apparatus, comprising:
a polymeric material provided in the form of a tubular body and comprising two or more polymers, the first polymer being different from the second polymer; and
a source of a water-containing fluid in communication with the polymeric material, the water-containing fluid imparted with a first moisture value, wherein the polymeric material is designed to remove moisture from the water-containing fluid to create a second fluid imparted with a second moisture value, and wherein the second moisture value is less than the first moisture value.

9. The apparatus of claim 8, wherein the polymeric material comprises two or more prepolymers, wherein the two or more prepolymers include at least one of:
a) diisocyanate blocks connected by urethane linkages to diols of variable chain length, or
b) a first alcohol or a carboxylic acid homopolymer block covalently bonded to a second alcohol or carboxylic acid homopolymer.

10. The apparatus of claim 8 further including a gas analyzer that measures at least one parameter of the second fluid.

11. The apparatus of claim 8, wherein the polymeric material comprises a block copolymer including a polyurethane block copolymer.

12. The apparatus of claim 8, wherein the water-containing fluid is provided in the form of a breath gas obtained from a patient.

13. The apparatus of claim 8, wherein the polymeric material comprises a composition including a blend of polymers, the composition including at least one of a polyurethane block copolymer or a polyester polyether block copolymer (COPE).

14. The apparatus of claim 8, wherein the polymeric material comprises a composition including two or more prepolymers, and wherein the two or more prepolymers include at least one of: (a) diisocyanate blocks connected by urethane linkages to diols of variable chain length, or (b) an alcohol or a carboxylic acid homopolymer block covalently bonded to another alcohol or carboxylic acid homopolymer.

15. A method of removing water content from a fluid, comprising:
collecting a water-containing fluid sample;
directing the water-containing fluid sample through a polymeric material to remove water from the water-containing fluid sample thereby creating a dehydrated fluid sample at least partially free of water contamination;
directing the dehydrated fluid sample out of the polymeric material and to an analyzer; and
evaluating the dehydrated fluid sample using the analyzer.

16. The method of claim 15, wherein the dehydrated fluid sample is substantially free of water contamination.

17. The method of claim 15, wherein the polymeric material is provided in the form of a polymer composition including a first polymer, and wherein the first polymer is selected from the group consisting of:
a polyurethane block copolymer;
a polyether block copolymer;
a polyester polyether block copolymer (COPE);
a diisocyanate block connected by urethane linkages to diols of variable chain length;
a first alcohol or a carboxylic acid homopolymer block covalently bonded to a second alcohol or carboxylic acid homopolymer; or combinations thereof.

18. The method of claim 15, wherein the water-containing fluid sample is provided as a water-containing breath gas, and wherein the method further includes steps of:
sampling water-containing breath gases of a patient by attaching a respiratory interface to the patient; and
placing an outlet of the respiratory interface into fluid communication with the polymeric material.

19. The method of claim 18, wherein the analyzer is configured to measure a concentration of at least one of oxygen, carbon dioxide, carbon monoxide, or nitrogen of the dehydrated fluid sample.

20. A method as in claim 15 wherein the polymeric material comprises at least a block copolymer comprising a polyester polyether block copolymer (COPE).
